(19) United States
(12) Patent Application Publication
(10) Pub. No.: US 2016/0184271 A1
(43) Pub. Date: Jun. 30, 2016

(54) Amino Acid Composition for Use in the Treatment of a PDD

(71) Applicant: N.V. Nutricia, Zoetermeer (NL)

(72) Inventor: Sofia Lopes Da Silva, Bunnik (NL)

(21) Appl. No.: 14/902,951

(22) PCT Filed: Jul. 4, 2014
(86) PCT No.: PCT/NL2014/050447

§ 371 Date: Jan. 5, 2016

(30) Foreign Application Priority Data
Jul. 5, 2013 (NL) PCT/NL2013/050505

(51) Int. Cl. A61K 31/4172; A61K 31/405; A23G 4/14; A61K 31/198

(52) U.S. Cl. CPC A61K 31/4172; A61K 31/198; A61K 31/405; A23L 1/3051; A23L 1/187; A23G 4/14; A23L 1/296; A23L 1/308; A23V 2002/00

(57) ABSTRACT

The invention relates to the use of at least one amino acid, selected from the group of histidine, lysine, and threonine in the manufacture of a composition for use in the treatment of a pervasive development disorder, a neurogenetic syndrome associated with a pervasive development disorder or a symptom of a pervasive development disorder. The invention further relates to a food product comprising histidine, lysine, and/or threonine, to a supplement comprising histidine, lysine, and/or threonine and to a kit of parts comprising histidine, lysine, and/or threonine.

The present invention relates to the use of an amino acid in the manufacture of a composition for use in the treatment of a pervasive development disorder (PDD), a syndrome associated with such disorder or a symptom of a PDD. Further, the invention relates to a product comprising one or more amino acids.

Among PDD, autistic spectrum disorders are complex developmental diseases, generally characterised by impairments in social functioning, communication and flexibility of thought and behaviour. Under DSM-IV, autistic spectrum disorders (ASDs) used to be categorized as follows (Johnson, C. P., and S. M. Myers. 2007. Identification and evaluation of children with autism spectrum disorders. Pediatrics 120:1183-215):
classic autism;
Asperger's syndrome: a disorder exhibiting the classical features of autism, with the exception that verbal and cognitive skills are higher;
PDD-NOS (Pervasive Developmental Disorder—Not Otherwise Specified), the remaining ASD, is described in the DSM-IV-TR as a sub-threshold diagnostic term used when a child demonstrates severe and pervasive impairments in reciprocal social skills associated with deficits in language skills or with the presence of stereotypic behaviours or restricted interests or activities but does not meet full criteria for classic autism or Asperger's syndrome. Under DSM-V these are all classified under ASD, which also includes childhood disintegrative disorder under DSM-V.

In addition, a number of neuro-genetic syndromes are known that are associated with ASDs. These include: Fragile X syndrome, Tuberous sclerosis, Neurofibromatosis type 1, disorders associated with phosphatase and tensin homo (PTEN) mutations, Rett syndrome, and Smith-Lemli-Opitz syndrome.

Treatment options of ASDs and syndromes associated with ASDs. are scarce. Long term Applied Behaviour Analytic intervention (ABA) has good effect size for language-related outcomes, but less effect on overall social functioning.

Gastrointestinal disorders and associated symptoms are commonly reported in individuals with ASDs, but key issues such as the cause and best treatment of these conditions are incompletely understood. Symptoms range from bloating and abdominal pain to loose stools and diarrhoea, and correlations with food intolerances, principally to milk (casein) and wheat (gluten), have been noted The strong correlation of gastrointestinal symptoms with autism severity indicates that persons with more severe autism are likely to have more severe gastrointestinal symptoms and vice versa.

WO 2008/071991A1 relates to the nutritional treatment of pervasive development disorders—such as autism—taking into account a potential role of food protein intolerance and/or food protein allergy in autism. It proposes to avoid dietary protein-intake completely or to exclude certain proteins in the diet and use free amino acids as a protein source. It is observed that the compositions of WO 2008/071991A1 can be used to overcome deficiencies caused by restrictions introduced in the diets of PDD patients and to improve their gastro intestinal health.

A treatment in accordance with WO 2008/071991A1 requires that the patient's diet is restricted with respect to consumption of proteins, which makes patient-compliance more difficult. Further, products based on free amino acids often suffer from aversive taste, which has a further adverse effect on patient compliance.

There remains a need for compositions for use in the treatment of PDD and syndromes associated with PDD. In particular, there is a need for a composition that has satisfactory patient compliance and that has a positive effect on a symptom characteristic for PDD, in particular a behavioural disturbance, for instance compulsive behaviour, low social interaction or traits of inflexibility.

It has now been found that it is possible to treat a trait of a PDD using one or more specific nutritional ingredients, without the need to avoid protein consumption, without the need to adhere to an elimination diet, or without the need to consume large amounts of free amino acids.

Accordingly, the invention relates to an amino acid for use in the treatment of a pervasive development disorder, a neurogenetic syndrome associated with a pervasive development disorder or symptom of a PDD.

Preferably, the invention relates to one or more amino acids for use in the treatment of ASD, preferably in the treatment of classic autism.

The amino acid(s) is/are selected from the group of arginine (arg), asparagine (asn), serine (ser), tyrosine (tyr), phenylalanine (phe), glycine (gly), histidine (his), lysine (lys), threonine (thr) and tryptophan (trp), in particular from the group of phenylalanine, glycine, histidine, lysine, threonine and tryptophan, more in particular from the group of histidine, lysine, threonine and tryptophan, more preferably from the group of histidine, lysine, and threonine.

The amino acid(s) is/are in particular intended to be used by a human.

In particular, the invention relates to the use of at least one amino acid, selected from the group of histidine, lysine, and threonine in the manufacture of a composition for use in the treatment of a pervasive development disorder, a neurogenetic syndrome associated with a pervasive development disorder or a symptom of a pervasive development disorder.

The invention further relates to a supplement comprising at least two amino acids selected form the group consisting of histidine, lysine, threonine and tryptophan, wherein the ratio [leu+ile+val]:[his+lys+thr+trp] is 1.0 or less, preferably 0.9 or less, preferably 0.8 or less, more preferably 0.6 or less

The invention further relates to a food product, preferably a food product selected from the group of confectionary products; nutritionally complete food products; desserts, in particular a pudding, yoghurt, vla, ice-cream, or milk-shake; beverages, in particular a fruit juice or milk; breakfasts, such as porridge, cereals; soups; and sauces, the food product comprising at least two amino acids selected from the group of threonine, histidine, lysine and tryptophan, wherein the total concentration of the amino acids selected from the group of threonine, histidine, lysine and tryptophan is in the range of 1-40 wt. %, in particular 5-40 wt. %, more in particular 10-40 wt. % of the total weight of the product.

The invention further relates to a kit of parts comprising a first package containing at least one amino acid selected from histidine, lysine, threonine and tryptophane, and a second package containing at least one other amino acid from the group of histidine, lysine, threonine and tryptophan, the contents of the packages of the kits of parts taken together having a ratio [leu+ile+val]:[his+lys+thr+trp] of 1.0 or less, preferably 0.9 or less, preferably of 0.8 or less, more preferably 0.6 or less.

The invention further relates to the use of a supplement or contents of a kit of parts according to the invention in a food product selected from the group of desserts, in particular a pudding, yoghurt, vla, ice-cream, or milk-shake; beverages, in particular a fruit juice or milk; breakfasts, such as porridge, cereals; soups; and sauces.

Further, the invention relates to a supplement, food product or kit of parts according to the invention for a medical use, in particular for a use in the treatment of a pervasive development disorder, a neurogenetic syndrome associated with a pervasive development disorder or a symptom of a pervasive development disorder. A supplement, food product or kit of parts for a medical use, is preferably for use in a daily dosage of the sum of histidine, lysine, threonine and tryptophan in the range of 1-20 g, in particular in the range of 2-10 g.

The invention further relates to a method of treating a subject diagnosed with a PDD or a subject having a neurogenic syndrome associated with a pervasive development or a subject with a symptom of a pervasive development disorder, the method comprising administering an effective amount of one or more of said amino acids, in particular one or more amino acids selected from the group of histidine, lysine and threonine, to said subject. The administration preferably is orally. The one or more amino acids are preferably administered using a composition, supplement, food product or contents of a kit of parts as described herein.

The inventors surprisingly found that changing the weight to weight ratio of daily intake of total branched chains amino acid sources (BCAA), i.e. the sum of leucine (leu) sources, isoleucine (ile) sources and valine (val) sources, to the daily intake of the sum of histidine (his) sources, lysine sources and threonine sources sources was effective in normalising both stereotypic behaviour and social interactive behaviour in mice suffering from cow milk allergy (CMA-mice). CMA-mice are an accepted animal model for evaluating the potential of a treatment of a PDD.

The inventors contemplate that in particular the weight to weight ratio [leu+ile+val]:[his+lys+thr+trp] in a daily diet play a role in a symptom typical for PDD. In an average regular diet, said ratio of [leu+ile+val]:[his+lys+thr+trp] is 1.3-1.4. The following table shows an example of amino acid sources consumption, provided by an exemplary daily food intake.

| | total amino | BCAA | lys + his + |
|---|---|---|---|
| Food: | acid | sources | thr + trp |
| 4.5 dL milk (1% lowfat) | 15.3 gram | 3.5 gram | 2.3 gram |
| 20 gram cheddar cheese | 5 gram | 1.1 gram | 0.8 gram |
| 4 slices of whole wheat bread | 18.2 gram | 1.1 gram | 0.7 gram |
| 75 gram meat/fish 80% lean beef | 18.8 gram | 3.2 gram | 3 gram |
| 50 grams pasta/rice/potato spaghetti | 6.5 gram | 1 gram | 0.6 gram |
| total | 63.8 gram | 9.9 gram | 7.4 gram |

The inventors found that a behavioural disturbance was effectively overcome when reducing the ratio of [leu+ile+val]:[his+lys+thr+trp], without needing to reduce the intake of the branched amino acids, also when no medication is taken. This was accomplished by increasing the intake of the sum of histidine (his), lysine and threonine. As shown in the Examples, changing said ratio was as effective as rap amycin, a medicament which has been reported to be effective in the treatment of symptoms of a PDD or neurogenetic syndrome with a PDD (e.g. Ehninger, D., S. Han, C. Shilyansky, Y. Zhou, W. Li, D. J. Kwiatkowski, V. Ramesh, and A. J. Silva. 2008. Reversal of learning deficits in a Tsc2+/− mouse model of tuberous sclerosis. Nat Med 14:843-8).

Rapamycin is primarily known as a mTor inhibitor. Thus, it was contemplated that an inhibitory effect on mTor may be relevant in achieving the observed effect on behaviour, even though—to the best knowledge of the inventors the amino acids for use in accordance with the invention are not known in the art as having an effect on mTor in vivo, nor is there any reason to assume that such an effect of amino acids on mTor would have an evolutionary benefit.

The amino acids for use according to the invention were tested for their effect on mTor, and although an inhibitory effect was observed, this effect was found to be far less effective than the inhibitory effect of rapamycin, typically at least about 10 times less inhibiting. For instance, 1 mM rapamycin had a comparable effect as 10 mM lysine.

Thus, the inhibitory in vitro effect of the amino acids for use according to the invention on mTOR is not considered the (sole) reason why said amino acids, upon dietary administration in vivo, were as effective as treatment with rapamycin

Further, various research suggests an opposite direction, namely that ASD patients have a relatively high occurrence in branched chain amino acid deficiency.

(e.g. Arnold, Hyman et al. J Autism Dev Disord 33(4): 449-54). In particular, lower levels were seen for leucine, valine and lysine. These amino acid deficiencies may well relate to poor nutrition secondary to unusual food preferences in children with autism that are often taste or texture based. E.g. Tirouvanziam, Obukhanych et al. (J Autism Dev Disord 42(5): 827-36), compared the plasma levels of amino acids in 27 children with ASD and in 20 neuro-typically developing children, Tirouvanziam, et al. observed reduced plasma levels of most polar neutral amino acids and leucine in children with ASD.

Also a publication of (Novarino, El-Fishawy et al. (Science 338(6105): 394-7) showed that ASD patients have lower levels of leucine. The accompanying press release from the University of Novarino and Gleeson reports that ASD patients may benefit from dietary supplementation with the branched chain amino acids (leucine, isoleucine, valine): http://health.ucsd.edu/news/releases/Pages/2012-09-06-autism-and-epilespsy.aspx.

It should be noted that the inventors found that leucine, valine and isoleucine have an mTOR activating effect in vitro, contrary to the amino acids that are considered to be useful in accordance with the present invention.

On the basis of these reports in the prior art one would have no reasons to expect that the amino acids for use in accordance with the invention, which have an inhibitory effect on mTOR would be effective. The invention allows to reduce the ratio [leu+ile+val]:[his+lys+thr+trp] by adding specific amino acids to a food product, e.g. a dessert, breakfast (cereals, porridge) a soup or a sauce, that is normally consumed by the patient, without needing to change the eating pattern the patient is used to. This is an important advantage, as subjects suffering from PDD, in particular ASD patients are usually characterized by strong traits of inflexibility. ASD patients, in particular, are known to have narrow food preferences and purported therapeutic diets may cause nutritionally inadequacies in this patient group. As it is not needed to reduce the intake of branched amino acids, the invention can be safely reduced to practice without risk of contributing to a deficiency in BCAA intake. Also the invention can safely be used by a subject whose diet contains protein.

As a further effect of an amino acid for use in accordance with the invention, it is contemplated that it may also alleviate a gastro-intestinal symptom in a patient suffering from a PDD.

The term “a” or “an” as used herein is defined as “at least one” unless specified otherwise.

When referring to a noun (e.g. a compound, an additive etc.) in the singular, the plural is meant to be included.

The term “or” as used herein is to be understood as “and/or” unless specified otherwise.

The term “amino acid” and the names of the specific amino acids (e.g. histidine) are used herein to refer the amino acid in any form (acceptable for consumption). The term “amino acid” specifically includes the monomeric form of the amino acid, such as the free amino acid (basic formula NH₂—R—COOH) or a salt thereof. Further, an amino acid may be present in bound form, i.e. form part of a larger compound. An amino acid may in particular form part of a peptide, which may be an oligopeptide, a polypeptide, or protein, or an amino acid ester. The term ‘protein’ includes proteins consisting of one or more polypeptides and compounds formed of a polypeptide and another moiety, such as a sugar moiety (glycoprotein) or a lipid (lipoprotein). When referred herein to an amino acid, proteinogenic and non-proteinogenic amino acid is meant, or its stereo-isomer. The human body has a capacity to convert D-isomers to L-isomers, and racemic mixtures of amino acids could in some cases be useful in the context of the invention. Generally, the use of the L-isomers is preferred.

The total of free amino acids, amino acids salts and amino acid in bound form is also referred to as ‘proteinaceous matter’.

As used herein pervasive development disorders, are generally as defined by DSM-V (http://www.dsm5.org/Pages/Default.aspx)

The criteria for ASD are:

An individual must meet criteria A, B, C and D:

A. Persistent deficits in social communication and social interaction across contexts, not accounted for by general developmental delays, and manifest by all 3 of the following:

1. Deficits in social-emotional reciprocity; ranging from abnormal social approach and failure of normal back and forth conversation through reduced sharing of interests, emotions, and affect and response to total lack of initiation of social interaction.

2. Deficits in nonverbal communicative behaviors used for social interaction; ranging from poorly integrated-verbal and nonverbal communication, through abnormalities in eye contact and body-language, or deficits in understanding and use of nonverbal communication, to total lack of facial expression or gestures.

3. Deficits in developing and maintaining relationships, appropriate to developmental level (beyond those with caregivers); ranging from difficulties adjusting behavior to suit different social contexts through difficulties in sharing imaginative play and in making friends to an apparent absence of interest in people.

B. Restricted, repetitive patterns of behavior, interests, or activities as manifested by at least two of the following:

1. Stereotyped or repetitive speech, motor movements, or use of objects; (such as simple motor stereotypies, echolalia, repetitive use of objects, or idiosyncratic phrases).

2. Excessive adherence to routines, ritualized patterns of verbal or nonverbal behavior, or excessive resistance to change; (such as motoric rituals, insistence on same route or food, repetitive questioning or extreme distress at small changes).

3. Highly restricted, fixated interests that are abnormal in intensity or focus; (such as strong attachment to or preoccupation with unusual objects, excessively circumscribed or perseverative interests).

4. Hyper-or hypo-reactivity to sensory input or unusual interest in sensory aspects of environment; (such as apparent indifference to pain/heat/cold, adverse response to specific sounds or textures, excessive smelling or touching of objects, fascination with lights or spinning objects).

C. Symptoms must be present in early childhood (but may not become fully manifest until social demands exceed limited capacities)

D. Symptoms together limit and impair everyday functioning

In an advantageous embodiment, the invention relates to an amino acid for use in the treatment of an autistic spectrum disorder (ASD).

In a specific preferred embodiment, the ASD is selected from the group of classic autism, Aperger's syndrome, PDD-NOS and childhood disintegrative disorder (as defined under DSM-IV, see also above).

In a specific embodiment, the invention relates to an amino acid for use in the treatment of a neurogenetic syndrome selected from the group of Fragile X syndrome, Tuberous sclerosis, Neurofibromatosis type 1, disorders associated with phosphatase and tensin homo (PTEN) mutations, Rett syndrome, and Smith-Lemli-Opitz syndrome.

In an advantageous embodiment, the amino acid is for use in the treatment of (abnormally) increased stereotypical behaviour, such as: restricted, repetitive patterns of behaviour, stereotyped or repetitive motor movements, or stereotyped or repetitive use of objects, excessive adherence to routines, ritualized patterns, excessive resistance to change. More specifically, the stereotypical behaviour may include motoric rituals, insistence on same route or food, extreme distress at small changes.

In an advantageous embodiment, the amino acid(s) is/are for use in the improvement of an abnormally low social interactive behaviour, such as abnormal social approach, total lack of initiation of social interaction, or difficulties in making friends

In principle, the composition can be any composition that is suitable for administration into the gastro-intestinal tract. Preferably, the composition is orally ingestible. Generally, a distinction can be made between food compositions in a classical sense (a product that nourishes, sustains or provides energy in enough volume to keep someone from being underfed), which products are intended to be consumed as such (e.g. confectionary products, desserts, breakfast) and food-supplements (which are intended be administered in addition to or together with a classical food product). The food supplement is preferably a product that is for administration in combination with a food product, such as a dessert or a fluid food product, for instance a beverage or fluid breakfast, more preferably a product that is added to, in particular mixed with, the food product.

With respect to the used amino acids, as mentioned above, in particular good results have been achieved with a diet wherein the sum of histidine, lysine, threonine and tryptophan is increased. This has in particular been achieved by increasing the daily intake of histidine, lysine and threonine. On the basis of these findings the inventors have identified tryptophan and other amino acids that are considered useful in a treatment according to the invention.

Phenylalanine and glycine are in particular considered in relation to their inhibitory effect of mTOR.

Arginine, asparagine, serine and tyrosine are capable of forming hydrogen bonds, which is also the case for histidine, lysine, threonine and tryptophan. Without being bound by theory it is contemplated that this may be physiologically relevant in relation to a treatment according to the invention.

Preferably, at least two amino acids are used in accordance with the invention, at least one of which is histidine, threonine or lysine.

In a preferred embodiment, at least two amino acids are used in accordance with the invention, at least one of which is histidine or threonine. More preferred, histidine and threonine are present in a composition according to the invention.

In a particularly preferred embodiment, at least three amino acids are used, preferably at least three of said amino acids are used selected from the group histidine, threonine, lysine phenylalanine, glycine and tryptophan (at least one of which is histidine, threonine or lysine), more preferably histidine, threonine and lysine.

The presence of threonine as free amino acid or salt thereof is in particular preferred for a positive effect on taste, in particular to compensate for a adverse taste of another free amino acid or salt thereof, such as free lysine or a salt thereof (fishy), or free tryptophan or a salt thereof (bitter).

Free histidine or a salt thereof is by some patients experienced as bitter or bitter-sweet, which may be a desirable taste effect, a neutral taste effect or an undesired taste effect, dependent on the patient.

In a specific embodiment, at least one of histidine, threonine or lysine is present as the major amino acid in the composition, i.e. is or are the most abundant amino acids in the composition. In such embodiment, the sum of histidine threonine and lysine preferably forms 50 wt. % or more of the total amino acids in the composition.

Advantageously, the composition comprising the amino acid(s) for use in accordance with the invention is administered to a subject whose total daily intake of proteinaceous matter, including the amino acid(s) for use in accordance with the invention, is characterised by a weight to weight ratio
[leu+ile+val]:[his+lys+thr+trp]
   of less than 1.0, preferably 0.9 or less, preferably of 0.8 or less, in particular of 0.7 or less more in particular of about 0.6 or less.

Branched chain amino acids are essential amino acids, at least for humans. Accordingly sources for these amino acids generally should form part of the (human) diet. Therefore the daily intake of proteinaceous matter (i.e. including intake via a composition according to the invention and other intake of proteinaceous matter) preferably provides a ratio of [leu+ile+val]:[his+lys+thr+trp] of at least 0.4.

A composition for use in accordance with the invention that comprises both histidine and threonine, usually comprises histidine and threonine in a weight to weight ratio in the range of 1:100 to 100:1, in particular in the range of 1:10 to 10:1. Said composition preferably comprises histidine and threonine in a weight to weight ratio of 1:1.2 or less, more preferably 1:3 or less, even more preferably 1:4.5 or less, and particularly preferred 1:5 or less, or even 1:6 or less. In a specific embodiment, said ratio is 1:5.5. In another specific embodiment, said ratio is 1:6.

A composition for use according to the invention that comprises both threonine and lysine usually comprises threonine and lysine in a weight to weight ratio in the range of 1:100 to 100:1, in particular in the range of 1:10 to 10:1. Said composition preferably comprises threonine and lysine in a weight to weight ratio of threonine:lysine at least 2:1, more preferably at least 3:1, even more preferably at least 3.5:1. In a specific embodiment said ratio is 4:1 or more. In another specific embodiment, said ratio is 5:1 or 6:1.

A high ratio threonine:lysine is in particular preferred for improved taste, in combination with satisfactory treatment of an PDD trait. Threonine is useful to mask a fishy taste, provided by lysine or another amino acid with adverse effect on taste.

The composition (for use) in accordance with the invention preferably provides a total daily dosage of at least two amino acids selected from the group of threonine, histidine, lysine and tryptophan in the range of 1-20 g/day, preferably 2-10 g/day. For an adult, the dosage preferably is at least 5 g/day. For a human adult, the dosage preferably is 10 g/day, or 9 g/day, or 8 g/day, or 7 g/day, or less. For a human child, the dosage preferably is at least 3 g/day. For a child, the dosage preferably is 6 g/day, or 5 g/day, or 4 g/day or less.

Preferably, the daily dosage of histidine, via a composition according to the invention, is 0-5 g, in particular 0.7-5 g, more preferably 1-4 g.

Preferably, the daily dosage of threonine, via a composition according to the invention, is 0-8 g, more preferably 1-8 g, in particular 2-7.5 g.

Preferably, the daily dosage of lysine, via a composition according to the invention, is 0-4 g, more preferably 0.8-3.5 g.

Preferably, the daily dosage of tryptophan, via a composition according to the invention, is 0-1 g, preferably 0.1-0.8 g, in particular 0.3-0.8 g.

The composition can be administered 2, 3, 4, or more times a day, and is preferably administered once a day. The total weight of the composition, administered per dosage, is preferably less than 400 grams, more preferably less than 200 grams, even more preferably less than 100 grams.

In case the composition is a supplement added to a food composition the total weight of the food composition plus supplement usually is 400 grams or less, preferably 200 grams or less. The volume per dosage usually is 400 ml or less, preferably mL or less.

Preferably, a daily dosage of arginine, asparagine, serine, tyrosine, phenylalanine and glycine, via a composition according to the invention is each independently in the range of 0-8 g, in particular in the range of 1-5 g.

The daily dosage of leucine, valine and isoleucine taken together, via a composition according to the invention, is preferably less than 2 g, preferably is 0-1 g.

The daily dosage of glutamic acid, via a composition according to the invention, typically is 0-0.7 g.

Histidine is preferably provided as a histidine HCl salt.

Lysine is preferably provided as a lysine HCl salt.

Threonine is preferably provided as a basic form of threonine

Tryptophan is preferably provided as a basic form of tryptophan

In a specific embodiment, the composition according to the invention is a supplement, preferably a powdered supplement, that can be added to a food product prior to consumption.

The total amino acid content of such a supplement is preferably 20-100 wt. %, in particular 20-90 wt. %, more in particular 20-75 wt. %.

Preferably the total amino acid content is at least 25 wt. %, in particular at least 30 wt. %.

Preferably, 50-100 wt. %, in particular 75-100 wt. %, more in particular 90-100 wt. % of the amino acid content is formed by one or more amino acids selected from the group of histidine, lysine, and threonine.

One or more amino acids selected from the group of phenylalanine, glycine, arginine, asparagine, serine, tyrosine, and tryptophan can be added to the supplement according to the invention.

If one or more additional amino acid are added (in addition to his/lys/thr), preferably, one or more amino acids selected from the group of phenylalanine, glycine, and tryptophan are added to the supplement according to the invention.

Preferably, 75-100 wt. %, in particular 90-100 wt. % of the amino acid content is formed by one or more amino acids selected from the group of histidine, lysine, threonine and tryptophan.

A supplement in accordance with the invention (possibly in the form of a kit of part), the sum of two, three or four of said amino acids selected from the group of threonine, histidine, lysine and tryptophan, when administered to a subject, preferably amounts in a total daily dosage of said amino acids via the supplement in the range of 1-20 g, more preferably 2-10 g; for an adult, preferably at least 5 g; for a human adult, preferably 10 g, or 9 g, or 8 g, or 7 g, or less; for a human child, preferably is 6 g, or 5 g, or 4 g or 3 g or less.

The supplement preferably comprises 0-30 wt. % histidine, more preferably 1-25 wt. % histidine, more preferably 2-20 wt. % histidine based on total weight of the supplement.

The supplement preferably comprises 0-25 wt. % lysine, more preferably 1-20 wt. % lysine, more preferably 2-15 wt. % based on total weight of the supplement.

The supplement preferably comprises at least 5 wt. % threonine, more preferably at least 6 wt. % threonine, in particular at least 10 wt. % threonine, more in particular at least 15 wt. % threonine, based on total weight of the supplement, The supplement preferably comprises 80 wt. % threonine or less, more preferably comprises 75 wt. % or less threonine, more preferably comprises 50 wt. % or less threonine, in particular 30 wt. % or less threonine, more in particular 20 wt. % or less threonine, based on total weight of the supplement.

The supplement usually comprises 0-15 wt. % tryptophan, preferably 0.5-10 wt. % tryptophan, in particular 1-5 wt. % tryptophan based on total weight of the supplement.

The total content of branched chain amino acids is preferably 0-15 wt. %, more preferably 0-10 wt. % of the supplement.

The total content of branched chain amino acids in a composition or supplement according to the invention is preferably 0-10 wt. %, based on total amino acids.

In an advantageous embodiment, the composition is essentially free of branched chain amino acids.

In an advantageous embodiment, the composition is essentially free of glutamine. It is contemplated that glutamine helps leucine getting into (brain) cells, thereby stimulating a potential adverse effect of leucine. If present, the glutamine content is preferably below 5 wt. %, more preferably below 1 wt. % of the supplement.

Tyrosine has a bitter taste and is difficult to dissolve. Therefore, in a specific embodiment, wherein this is a serious issue, it is advantageous that the composition is essentially free of tyrosine, in particular in a supplement for use in a food product, wherein it is to be dissolved. Alternatively, if tyrosine is present, it is preferred to mask its taste with a suitable flavour, such as a citrus flavour, or to include threonine in a relatively high amount.

Phenylalanine competes with tryptophan in passing the blood brain barrier, and is therefore considered less preferred for use in combination with tryptophan. Evidently, the composition should be free of phenylalanine if the composition should be suitable for administration to a PKU-patient.

As indicated above amino acids can be provided in a free form or salt thereof, or as part of a larger molecule, such as a peptide. Advantageously, at least part of the amino acid(s) for use in accordance with the invention is provided in monomeric form (free amino acid or a salt thereof). Preferably at least 25 wt. %, in particular at least 50 wt. % more in particular at least 75 wt. % of the amino acid(s) for use in accordance with the invention is provided in the composition as a free amino acid or a salt of a free amino acid. It is in particular preferred that at least 25 wt. %, in particular at least 50 wt. % more in particular at least 75 wt. % of the amino acids selected from the group of histidine, lysine, threonine and tryptophan in the composition is free amino acid or a salt thereof.

Advantageously, the weight to weight ratio of [leu+ile+val] in monomeric form to [his+lys+thr+trp+arg+asn+ser+tyr+phe+gly] in monomeric form in a composition, in particular a supplement, according to the invention is less than 1, preferably 0.8 or less, more preferably 0.7 or less, more preferably 0.6 or less, in particular 0.5 or less, in particular 0.4 or less, or 0.2 or less.

The ratio of [leu+ile+val]:[his+lys+thr+trp] in a composition (i.e. said ratio of amino acids in any form), in particular a supplement, according to the invention is preferably less than 1, more preferably is 0.8 or less, more preferably 0.7 or less, even more preferably 0.6 or less, in particular 0.5 or less, or in particular 0.3 or less.

The ratio of [leu+ile+val]:[his+lys+thr] in a composition, in particular a supplement, according to the invention preferably is less than 1.25, preferably 1 or less, preferably 0.9 or less, more preferably 0.8 or less, in particular 0.6 or less, more in particular 0.3 or less.

In a specific embodiment, the ratio of the weight of tryptophan to that of the branched amino acids leucine, isoleucine and valine in a composition of the invention is at least 0.22, preferably 0.23 to 20 and most preferably 0.28 to 4.

In principle, the composition, in particular a supplement, may essentially consist of amino acids.

The composition, in particular a supplement, according to the invention may essentially consist of coated amino acids. Examples of suitable coating materials include solid lipids, emulsifiers, carbohydrates and proteins. Many techniques known in the art are available for coating amino acids.

Preferably, the composition contains one or more further food-grade ingredients, typically in a total concentration of up to 80 wt. % of the composition, preferably in a total concentration of 70 wt. % or less, or 50 wt. % or less, more preferably in a total concentration of 30 wt. % or less, 20 wt. % or less, or even 10 wt. % or less. The food-grade ingredients are preferably selected from the group of dietary fibres, amino acids other than histidine, lysine, threonine, tryptophan (provided that the BCAA levels remain sufficiently low), minerals, digestible carbohydrates, vitamins and flavours.

If present, the protein content of the supplement, usually is at least 1 wt. %, in particular at least 5 wt. %, more in particular at least 15 wt. %. The protein content usually is 40 wt. % or less, in particular 30 wt. % or less.

If present, the digestible carbohydrate content of the supplement, usually is at least 1 wt. %, in particular at least 5 wt. %, preferably at least 30 wt. %, in particular at least 40 wt. %. The carbohydrate content usually is 80 wt. % or less, preferably 70 wt. % or less, in particular about 60 wt. % or less. The presence, of digestible carbohydrate in a relatively high amount is in particular preferred for masking the taste of free amino acids or salts thereof.

In an embodiment, the supplement is packaged in a dosage unit packaging, in particular a sachet, preferably containing 2 to 50 grams, preferably 3 to 20 grams, or more preferably 4-15 grams of the supplement. In an alternative embodiment, the supplement is present in a container from which multiple dosages can be taken, for instance using a spoon.

In an embodiment, the supplement is package as a kit of parts, comprising to or more separately packaged ingredients for the supplement, the packages containing a composition having a different amino acid(s) content, and the combined contents of the package forming a supplement according to the invention. It is also envisaged that the kit of parts comprises a package containing a food product, for instance a beverage or a dessert, to which the contents of one or more other packages (providing the amino acids) can be added.

The edible contents of a kit of parts preferably have an amino acid composition as described herein for a supplement of the invention.

The supplement or kit of parts may in particular be added to a dessert, in particular pudding, yoghurt, vla, ice-cream, a milk-shake, a beverage, such as fruit juice or milk, a breakfast, e.g. porridge, cereals. The supplement can be added shortly before consumption by the patient, or someone taking care of the patient.

In an alternative embodiment, the composition is a food product in a classical sense. Such food products include desserts, breakfasts, nutritionally complete food products and confectionary products.

A preferred food product is a confectionary product selected from the group of food-bar (such as granola bars, candy bars), sweeties and cookies. The total concentration of the amino acids suitable for a use according to the invention, in the food product preferably is in the range of 5-40 wt. %, in particular 10-40 wt. %, of the total weight of the product. Preferably, the total concentration of the amino acids selected from the group of threonine, histidine, lysine and tryptophan is in the range of 20-40 wt. % of the total weight of the product. The ratio [leu+ile+val]:[his+lys+thr+trp] of the food product is generally such that it is suitable to reduce said ratio in the total intake of amino acids by a subject consuming a regular daily diet (wherein said ratio is about 1.3). Thus, the food composition usually has a weight to weight ratio [leu+ile+val]:[his+lys+thr+trp] below 1, preferably of 0.8 or less, more in particular of 0.7 or less.

The protein in a composition according to the invention can be selected from any food-grade protein. Preferably the protein is selected from pea protein, quinoa protein and soy protein. These proteins were found to have a relatively low ratio [leu+ile+val]:[his+lys+thr+trp], and thus such a protein is considered to contribute to an advantageous effect on a PDD patient.

Soy protein, is in particular useful for a composition intended for the treatment of a subject allergic to cow's milk or gluten.

Further, collagen (constituent of gelatin) may advantageously be present in some embodiments, e.g. in a powder for a pudding or a confectionary products, such as a wine gum.

In particular, good results have been achieved with a composition that is essentially free of whey protein, caseinate and/or casein.

The digestible carbohydrate in a composition according to the invention can be selected from any food grade digestible carbohydrate. Preferably, the carbohydrate fraction in the composition provides a glycaemic index (GI) of less than 80. It is contemplated that this has a positive effect on an autistic trait, potentially via reducing insulin release, which release increases upon the consumption of high glycemic index carbs, transiently activates mTOR via the IGF receptor.

As used herein, the GI is the measure of its ability to raise postprandial glucose concentrations, using a method as described in WO 2010/137979. The skilled person will be able to formulate a composition with such a GI, based on common general knowledge and the information disclosed herein and in the cited prior art.

Preferred carbohydrates for providing a relatively low GI are maltodextrins, either heat treated or not in order to decrease glucose index to value of less than 80. Other examples of carbohydrates that contribute to a lower GI than glucose are isomaltulose, fructose, galactose, lactose and threalose.

Optionally, rapidly available carbohydrates are included having relatively low sweetness, preferably less than 80% of the sweetness of sucrose. A preferred example of such rapidly available carbohydrates is glucose.

A composition, or a supplement, according to the invention may comprise a dietary fibre. If present, the fibre content is usually in the range of 1 to 10 wt. % of the composition, in particular 2 to 5 wt. %. Preferred fibres are in particular fibres that are not substantially fermented in the human gastro-intestinal system, such as GOS and cellulosic fibres. Such fibre is preferably an immune modulatory fibre.

In a specific embodiment a composition according to the invention, comprise less than 5 wt. % fermentable dietary fibre, in particular less than 2 wt. % fermentable dietary fibre, more in particular less than 1 wt. % fermentable dietary fibre.

In a specific embodiment, a composition according to the invention comprises tryptophan and less than 5 wt. % cereal fibre, in particular less than 2 wt. % cereal fibre, more in particular less than 1 wt. % cereal fibre.

In a specific embodiment, a composition according to the invention comprises dietary fibre, in particular cereal dietary fibre, more in particular rice dietary fibre and the ratio of tryptophan to the sum of leucine isoleucine and valine is 0-0.21, in particular 0-0.15.

In a specific embodiment, a composition according to the invention comprises dietary fibre and more than 6 mg/g tryptophan, based on total weight, in particular more than 7 mg/g tryptophan, based on total weight.

In an embodiment, the composition of the invention comprises one or more additional components, in particular one or more components selected from flavours (other than carbohydrates), aromas, minerals and vitamins. Suitable components and concentrations can be based on general known dietary recommendations or based on the information provided in PCT/NL2012/050483, of which the contents in the section ‘Other components” starting at page 26, line 26 till page 32 line 25 are incorporated herein by reference.

A preferred flavour is selected from the group of citrus flavours. Such flavour has been found particularly suitable to mask a bitter taste of an amino acid.

A composition according to the invention can be manufactured in a manner known per se. The ingredients for a powder can be dry-blended according to methods known in the art. In an alternative method the ingredients are dissolved in water and spray dried according methods known in the art.

The invention is now illustrated by the following examples.

### EXAMPLE 1

Core features of autism (novelty induced stereotypic behaviour and reduced social interactive behaviour) were induced in mice by rendering them allergic to whey protein upon 5 weekly rounds of sensitization with whey and cholera toxin (CT) or CT alone (control), an I.g. challenge with whey (intragastrically administered whey) 6 days after the 5^{th} sensitation, and evaluation of social behaviour 7 days after the 5^{th} sensitation.

In this mouse cow's milk allergic (CMA) model, two diets were compared for their effects on social interactive behaviour and novelty induced stereotypic behaviour. In the control AIN-93 G based diet, the protein fraction was substituted by free amino acids (or salt thereof) in amounts as present in soy protein (in the Figures depicted as ‘soy a.a.’). In the active diet, this ratio of amino acids was changed: the branched chain amino acids (in italics in the following Table) leucine, isoleucine, and valine, were decreased and histidine, lysine and threonine were increased (underlined in the following). The proteinaceous matter consisted of free amino acids (or salt thereof). The ratio [leu+ile+val]:[his+lys+thre+trp] in the control diet was as in a regular average daily diet (i.e. ratio of 1.3-1.4). The ratio [leu+ile+val]:[his+lys+thre+trp] in the active diet was about twice as low (i.e. ratio of 0.6).

| | | Diet according to |
|---|---|---|
| | Control diet | the invention |
| Amino acid | (g/kg diet) | (g/kg diet) |
| Alanine | 7.68 | 7.77 |
| Arginine | 13.31 | 13.47 |
| asparigine | 0.00 | 0.00 |
| Aspartic acid | 20.13 | 20.37 |
| Cysteine | 2.05 | 2.07 |
| glutamine | 13.50 | 13.66 |
| Glutamic acid | 21.47 | 21.73 |
| Glycine | 7.51 | 7.60 |
| Histidine | 4.27 | 6.32 |
| Isoleucine | 8.70 | 5.60 |
| Leucine | 14.25 | 9.16 |
| Lysine | 9.55 | 14.17 |
| Methionine | 5.00 | 5.00 |
| Phenylalanine | 9.30 | 9.41 |
| Proline | 8.36 | 8.46 |
| Serine | 8.79 | 8.89 |
| Threonine | 6.65 | 9.83 |
| Tryptophan | 2.13 | 2.16 |
| Tyrosine | 6.65 | 6.73 |
| Valine | 8.70 | 5.60 |
| total | 178.00 | 178.00 |

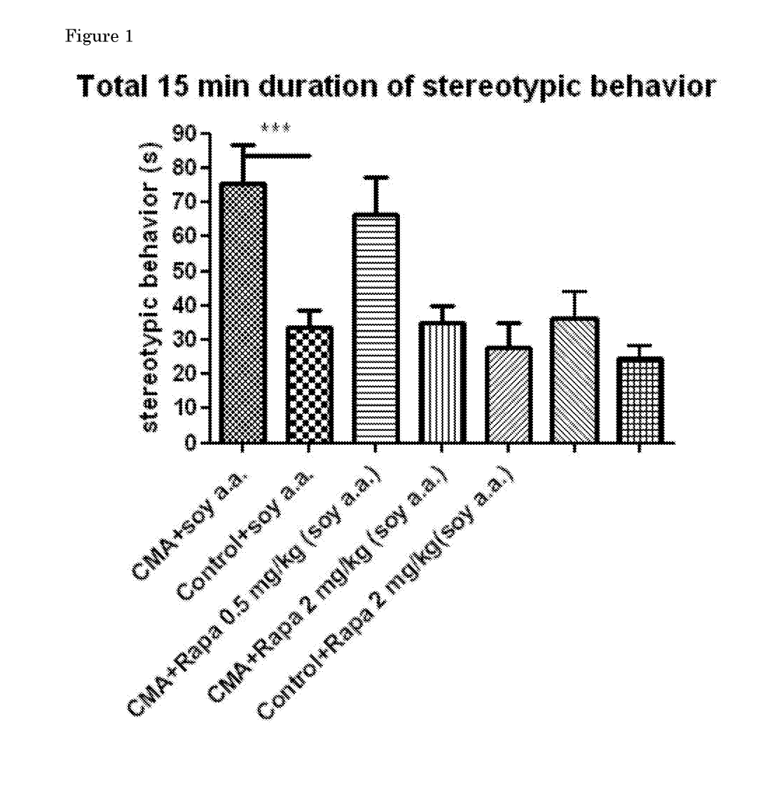

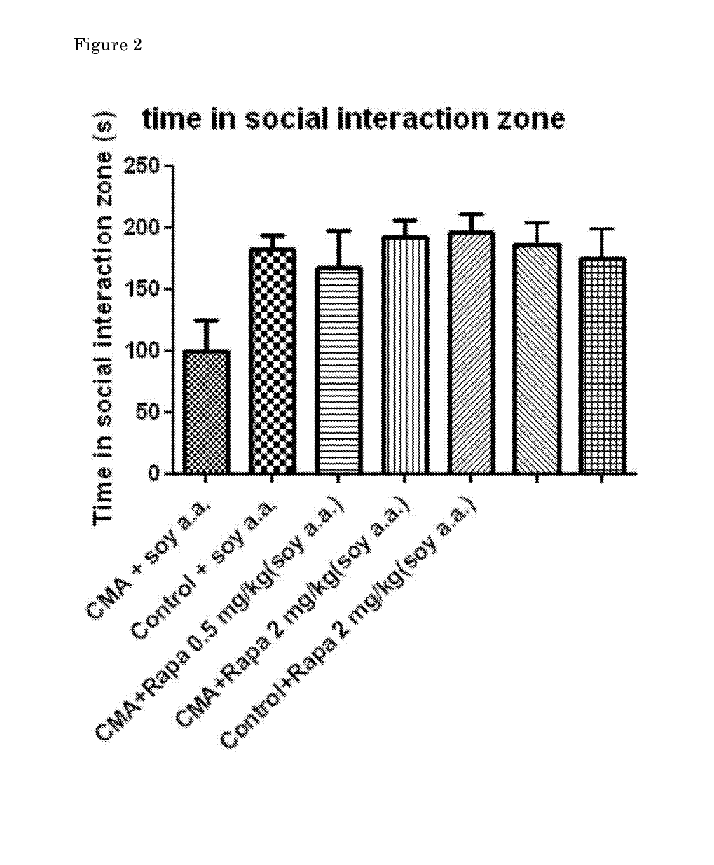

Effects of the treatment according to the invention was compared with the effect of an intraperitonatal injection with rapamycine (rapa) in a daily dosage of 0.5 or 2 mg/kg body weight on the following aspects: stereotypic behaviour (excessive grooming) and social interaction. The results are shown in FIGS. 1 and 2 respectively.

### EXAMPLE 2

### Preferred Embodiment

Per table spoon of 15 ml volume that will comprise 10 g of powder the sum of the amino acids for use according to the invention is 4 g, preferably at least two amino acids are present selected from the group of: histidine, threonine and lysine. More preferably histidine and threonine are present. The powder contains 6 g of digestible carbohydrates.

Preferably, the histidine:threonine weight ratio is 1:1.2-3.

If histidine, threonine and lysine are present the weight ratio histidine:threonine:lysine preferably is 1-1.8:2-3.5:1.

### EXAMPLE 3

### Preferred Embodiment

A powder, preferably in a sachet of 10-20 g, wherein the sum of the amino acids for use according to the invention is 8 g, wherein preferably at least two amino acids are present selected from the group of: histidine, threonine, lysine and tryptophan.

More preferably histidine and threonine are present.

Preferably, the powder contains 6 g of digestible carbohydrates.

Preferably, the histidine:threonine weight ratio is 1:4.5.

If histidine, threonine and lysine are present the weight ratio histidine:threonine:lysine is preferably 1:6:1.

### EXAMPLE 4

### Powders Useful for Fortifying a Food Product

Ingredients as used to manufacture 10 g of powder:

3-5 g of the sum of amino acids, preferably the amino acids as in Example 2 or 3 and in the same ratio as specified in Example 2 or 3.
3-7 g digestible carbohydrates
0-4 g other food grade components (proteins, minerals, vitamins)

Preferably the protein is pea protein or quinoa protein.

The powder can be added to a food product, such as a dessert, in particular pudding, yoghurt, vla, ice-cream, a milk-shake, a beverage, such as fruit juice or milk, a breakfast, e.g. porridge, cereals, a soup, or a sauce, e.g. shortly before use.

### EXAMPLE 5

### Food-Bar

Bar of 20 g comprising 8 g of the sum of Threonie+Histidine+Lysine, preferably in a ratio a described in Example 2 or 3.

The amino acids are coated by methods known in the art (e.g. WO2008/130236) and mixed with usual ingredients for a food bar, such as granola-bar ingredients, and formed in a bar-shape.

### EXAMPLE 6

### Cookie

Comprising 5-40 wt. % of the sum of Threonie+Histidine+Lysine, preferably in a ratio a described in Example 2 or 3.

The amino acids are coated by methods known in the art (e.g. WO2008/130236) and mixed with usual ingredients for a cookie, such as butter, salt, sugar, preferably soy flower, vanilla flavour, citrus flavour, and water in effective amounts to prepare a cookie dough. The cookie dough is shaped and baked to form the cookie.

### EXAMPLE 7

### Sweetie (Wine Gum)

Comprising 5-40 wt. % of the sum of Threonie+Histidine+Lysine, preferably in a ratio a described in Example 2 or 3.

The amino acids have been coated by methods known in the art (e.g. WO2008/130236) and are mixed with usually ingredients for the sweetie, such as glucose syrup, sugar, acid treated starch (from soy origin), gelatine, water, food acids, flavors, vegetable fat, colors, and formed into the sweetie shape.

### EXAMPLE 8

### Powder for Preparing a Ready to Eat Pudding

As described in Example 2 or 3, with exception that at least 15 wt % of gelatin is present and the total concentration of protein+gelatin is 15-40 wt. %.

### EXAMPLE 9

### Nutritionally Complete Liquid for Children

The liquid comprises per 100 ml:
The sum of Thr, Lys and His 4.0 g. preferably in a ratio as defined in Example 2 or 3.
Protein derived from pea, quinoa or soy: 0.4 g
Digestible carbohydrates: 10.4 g

### Fat: 3.0 g

Dietary fibre 0.8 g
Minerals: Na 60 mg. K 110, Cl 95, Ca 60, P 50 mg, Mg 11, Fe 1.0 mg, Zn, 1.0, Cu 90 ug, Mn 0.15 mg, F 0.07 mg, Mo 4 ug, Se 3 ug, Cr 3.5 ug, I 10 ug
Vitamins, A 41 ug RE, vit D 1 ug, vit E 1.3 mg a-TE, vit K 4 ug, B1 0.15 mg, B2 0.16 mg, B3 1.1 mg NE, pantothenic acid, 0.33 mg, B6 0.12 mg, B11 15 ug, B12 0.17 ug, biotin 4 ug, vit C 10 mg,
Miscellaneous: choline 20 mg

### EXAMPLE 10

### Nutritionally Complete Liquid for Adult

The liquid comprises per 200 ml:
The sum of Thr, Lys and His 8 to 9.0 g. preferably in a ratio [leu+ile+val] to [his+lys+thre+trp] of less than 1, more preferably of about 0.6.
Protein derived from pea, quinoa or soy: 0.4 g
Digestible carbohydrates: 10.4 g

### Fat: 3.0 g

Dietary fibre 0.8 g
Minerals: Na 60 mg. K 110, Cl 95, Ca 60, P 50 mg, Mg 11, Fe 1.0 mg, Zn, 1.0, Cu 90 ug, Mn 0.15 mg, F 0.07 mg, Mo 4 ug, Se 3 ug, Cr 3.5 ug, I 10 ug
Vitamins, A 41 ug RE, vit D 1 ug, vit E 1.3 mg a-TE, vit K 4 ug, B1 0.15 mg, B2 0.16 mg, B3 1.1 mg NE, pantothenic acid, 0.33 mg, B6 0.12 mg, B11 15 ug, B12 0.17 ug, biotin 4 ug, vit C 10 mg,
Miscellaneous: choline 20 mg

1. Use of at least one amino acid, selected from the group of histidine, lysine, and threonine in the manufacture of a composition for use in the treatment of a pervasive development disorder, a neurogenetic syndrome associated with a pervasive development disorder or a symptom of a pervasive development disorder.

2. Use according to claim 1, wherein the composition further comprises at least one amino acid selected from the group of phenylalanine, glycine and tryptophan, preferably tryptophan.

3. Use according to claim 1 or 2, wherein the composition has a weight to weight ratio [leu+ile+val]:[his+lys+thr+trp] of less than 1.0, preferably of 0-0.80, more preferably of 0-0.70, or even more preferably of 0-0.60, in particular of 0-0.3.

4. Use according to any of the preceding claims, wherein the amino acid is for use in the treatment of a pervasive development disorder, wherein said disorder is an autistic spectrum disorder, preferably an autistic spectrum disorder selected from the group of classic autism, Aperger's syndrome, childhood disintegrative disorder and PDD-NOS.

5. Use according to any of claims 1 to 3, wherein the amino acid is for use in the treatment of a neurogenetic syndrome selected from the group of Fragile X syndrome, Tuberous sclerosis, Neurofibromatosis type 1, disorders associated with phosphatase and tensin homo (PTEN) mutations, Rett syndrome, and Smith-Lemli-Opitz syndrome.

6. Use according to any of the preceding claims, wherein the amino acid is for use in the treatment of abnormal stereotypical behaviour, in particular for use in the treatment of restricted, repetitive patterns of behaviour, stereotyped or repetitive motor movements, or stereotyped or repetitive use of objects, excessive adherence to routines, ritualized patterns, or excessive resistance to change.

7. Use according to any of the preceding claims, wherein the amino acid is for improving abnormally low social interaction, in particular for improving abnormal social approach, total lack of initiation of social interaction, or difficulties in making friends.

8. Use according to any of the preceding claims, wherein at least one of histidine, threonine or lysine is present as the major amino acid in said composition.

9. Use according to any of the preceding claims, wherein at least two amino acids are used, at least one of which is histidine, threonine or lysine.

10. Use according to any of the preceding claims, wherein at least two amino acids are used, at least one of which is histidine or threonine.

11. Use according to any of the preceding claims, wherein at least three of said amino acids are used selected from the group histidine, threonine, lysine phenylalanine, glycine and tryptophan (at least one of which is histidine, threonine or lysine), preferably histidine, threonine and lysine.

12. Use according to any of the preceding claims, wherein the composition is to be administered to a subject whose total daily intake of proteinaceous matter, including proteinaceous matter provided by said composition, is characterised by a weight to weight ratio
[leu+ile+val]:[his+lys+thr+trp]
of less than 1.0, preferably of less than 0.9, in particular in the range of 0.4-0.8, more in particular in the range of 0.5-0.7.

13. Use according to any of the preceding claims, wherein the composition is to be administered in an effective amount to provide a daily dosage of the sum of the amino acids selected from the group of threonine, histidine, lysine and tryptophan from said composition in the range of 1 to 20 g/day, preferably 2-10 g/day, preferably in the range of 4-10 g/day for an adult, more preferably 5-9 g/day for an adult, respectively 3-6 g/day for a child.

14. Use according to any of the preceding claims wherein the composition comprises histidine and threonine, preferably in a weight to weight ratio histidine:threonine of 1:1.2 or less, in particular 1:4.5 or less, preferably of 1:6 or less.

15. Use according to any of the preceding claims, wherein the composition comprises threonine and lysine in a weight to weight ratio of threonine:lysine of at least 2:1, in particular of at least 4:1.

16. Use according to any of the preceding claims, wherein at least 25 wt. %, in particular at least 50 wt. % more in particular at least 75 wt. % of the amino acids selected from the group of histidine, lysine, threonine and tryptophan is provided in the composition as a free amino acid or a salt of a free amino acid.

17. Use according to any of the preceding claims, wherein the composition is to be consumed by a subject whose diet contains protein.

18. Use according to any of the preceding claims, wherein the composition is to be consumed by a human.

19. Use according to any of the preceding claims, wherein the composition is a food supplement to be added to a food product prior to consumption.

20. Use according to any of the preceding claims, wherein the composition is a food supplement, which food supplement comprises
0-30 wt. % histidine, preferably 1-25 wt. % histidine, more preferably 2-20 wt. % histidine, based on total weight of the supplement;
0-25 wt. % lysine, preferably 1-20 wt. % lysine, more preferably 2-15 wt. % based on total weight of the supplement;
0-80 wt. % threonine, preferably 5-30 wt. % threonine, in particular 6-20 wt. % threonine, based on total weight of the supplement;
0-15 wt. % tryptophan, preferably 1-5 wt. % tryptophan, based on total weight of the supplement;
the sum of [histidine+lysine+threonine+tryptophan] forming at least 20 wt. % of the supplement, preferably 30-80 wt. % of the supplement;
the weight to weight ratio
[leu+ile+val]:[his+lys+thr+trp]
in the supplement being 0-0.9, in particular 0-0.8, more in particular 0-0.5, preferably 0-0.3;
which supplement may further comprise one or more other food-grade ingredients, which together form up to 80 wt. % of the composition,
which food-grade ingredients are preferably selected from the group of dietary fibres, amino acids other than histidine, lysine, threonine, tryptophan, minerals, digestible carbohydrates, vitamins and flavours.

21. Use according to any of the claims 1-20, wherein the composition is a food product, preferably a food product selected from the group of confectionary products; nutritionally complete food products; desserts, in particular a pudding, yoghurt, vla, ice-cream, or milk-shake; beverages, in particular a fruit juice or milk; breakfasts, such as porridge, cereals; soups; and sauces, wherein the total concentration of the amino acids selected from the group of threonine, histidine, lysine and tryptophan is in the range of 1-40 wt. %, in particular 5-40 wt. %, more in particular 10-40 wt. % of the total weight of the product.

22. Supplement comprising at least two amino acids selected form the group consisting of histidine, lysine, threonine and tryptophan, wherein the ratio [leu+ile+val]:[his+lys+thr+trp] is 1.0 or less, preferably 0.9 or less, preferably 0.8 or less, more preferably 0.6 or less.

23. Supplement according to claim 22, wherein the supplement comprises
0-30 wt. % histidine, preferably 1-25 wt. % histidine more preferably 2-20 wt. % histidine, based on total weight of the supplement;
0-25 wt. % lysine, preferably 1-20 wt. % lysine, more preferably 2-15 wt. % based on total weight of the supplement;
0-80 wt. % threonine, preferably 5-30 wt. % threonine, in particular 6-20 wt. % threonine, based on total weight of the supplement;
0-15 wt. % tryptophan, preferably 1-5 wt. % tryptophan, based on total weight of the supplement;
the sum of [histidine+lysine+threonine+tryptophan] forming at least 20 wt. % of the supplement, preferably 30-80 wt. % of the supplement.

24. Supplement according to claim 22 or 23, which supplement comprise one or more other food-grade ingredients than histidine, lysine, threonine and tryptophan, which other food-grade ingredients together form up to 80 wt. % of the composition, which food-grade ingredients selected from the group of dietary fibres, amino acids other than histidine, lysine, threonine and tryptophan, minerals, digestible carbohydrates, vitamins and flavours.

25. Supplement according to any of the claims 22-24, comprising 15-40 wt. % of protein.

26. Supplement according to any of the claims 22-25, comprising 20-80 wt. % digestible carbohydrates, in particular 30-60 wt. % digestible carbohydrates.

27. Supplement according to any of the claims 22-26, comprising histidine and threonine in a weight to weight ratio histidine:threonine of 1:1.2 or less, in particular 1:4.5 or less, more in particular of 1:6 or less.

28. Supplement according to any of the claims 22-27, comprising threonine and lysine in a weight to weight ratio of threonine:lysine of at least 2:1, in particular of at least 4:1.

29. Supplement according to any of the claims 22-28, wherein at least 25 wt. %, in particular at least 50 wt. % more in particular at least 75 wt. % of the amino acids selected from the group of histidine, lysine, threonine and tryptophan is provided in the composition as a free amino acid or a salt of a free amino acid.

30. Use of a supplement according to any of the claims 22-29 in a food product selected from the group of desserts, in particular a pudding, yoghurt, vla, ice-cream, or milk-shake; beverages, in particular a fruit juice or milk; breakfasts, such as porridge, cereals; soups; and sauces.

31. Food product, preferably a food product selected from the group of confectionary products; nutritionally complete food products; desserts, in particular a pudding, yoghurt, vla, ice-cream, or milk-shake; beverages, in particular a fruit juice or milk; breakfasts, such as porridge, cereals; soups; and sauces, the food product comprising at least two amino acids selected from the group of threonine, histidine, lysine and tryptophan, wherein the total concentration of the amino acids selected from the group of threonine, histidine, lysine and tryptophan is in the range of 1-40 wt. %, in particular 5-40 wt. %, more in particular 10-40 wt. % of the total weight of the product.

32. Supplement according to any of the claims 22-29 or a food product according to claim 31, comprising per dosage unit:
1-4 g histidine
2-7.5 g threonine
0.8-3.5 g lysine
0-0.8 g tryptophan
in a total amount of 1-20 g, in particular 2-10 g.

33. Supplement or food product according to any of the claim 22-29, 31 or 32, wherein the weight to weight ratio histidine:threonine is 1:4 to 1:10, in particular about 1:6.

34. Supplement or food product according to any of the claim 22-29, or 31-33, wherein the weight to weight ratio lysine:threonine is 1:4 to 1:10, in particular about 1:6.

35. Kit of parts comprising a first package containing at least one amino acid selected from histidine, lysine, threonine and tryptophane, and a second package containing at least one other amino acid from the group of histidine, lysine, threonine and tryptophan, the contents of the packages of the kits of partxs taken together having a ratio [leu+ile+val]:[his+lys+thr+trp] of 1.0 or less, preferably of 0.9 or less, preferably of 0.8 or less, more preferably 0.6 or less.

36. Kit of parts according to claim 35, wherein the kit of part contains
0-30 wt. % histidine, preferably 1-25 wt. % histidine more preferably
2-20 wt. % histidine, based on total weight of the edible contents of the kit of parts;
0-25 wt. % lysine, preferably 1-20 wt. % lysine, more preferably 2-15 wt. % based on total weight of the edible contents of the kit of parts;
0-80 wt. % threonine, preferably 5-30 wt. % threonine, in particular 6-20 wt. % threonine, based on total weight of the edible contents of the kit of parts;
0-15 wt. % tryptophan, preferably 1-5 wt. % tryptophan, based on total weight of the edible contents of the kit of parts;
the sum of [histidine+lysine+threonine+tryptophan] forming at least 20 wt. % of the supplement, preferably 30-80 wt. % of the kit of parts.

37. Kit of parts according to claim 35 or 36, comprising histidine and threonine in a weight to weight ratio histidine:threonine of 1:1.2 or less, in particular 1:4.5 or less, more in particular of 1:6 or less.

38. Kit of parts according to any of the claims 35-37, comprising threonine and lysine in a weight to weight ratio of threonine:lysine of at least 2:1, in particular of at least 4:1.

39. Kit of parts according to any of the claims 35-38, wherein at least 25 wt. %, in particular at least 50 wt. % more in particular at least 75 wt. % of the amino acids selected from the group of histidine, lysine, threonine and tryptophan is provided in the composition as a free amino acid or a salt of a free amino acid.

40. Use of the contents of a kit of parts according to any of the claims 35-39 in a food product selected from the group of desserts, in particular a pudding, yoghurt, vla, ice-cream, or milk-shake; beverages, in particular a fruit juice or milk; breakfasts, such as porridge, cereals; soups; and sauces

41. Supplement, food product or kit of parts according to any of the claim 22-29, or 31-39, wherein the supplement, food product or contents of the kit of parts comprise less than 5 wt. % fermentable dietary fibre, in particular less than 2 wt. % fermentable dietary fibre, more in particular less than 1 wt. % fermentable dietary fibre.

42. Supplement, food product or kit of parts according to any of the claim 22-29, or 31-41, wherein the supplement, food product or contents of the kit of parts comprises tryptophan and less than 5 wt. % cereal fibre, in particular less than 2 wt. % cereal fibre, more in particular less than 1 wt. % cereal fibre.

43. Supplement, food product or kit of parts according to any of the claim 22-29, or 31-42 wherein the supplement, food product or contents of the kit of parts comprises dietary fibre, in particular cereal dietary fibre, more in particular rice dietary fibre and the ratio of tryptophan to the sum of leucine isoleucine and valine is 0-0.21, in particular 0-0.15.

44. Supplement, food product or kit of parts according to any of the claim 22-29, or 31-43 wherein the supplement, food product or contents of the kit of parts comprises dietary fibre and more than 6 mg/g tryptophan, based on total weight, in particular more than 7 mg/g tryptophan, based on total weight.

45. Supplement, food product or kit of parts according to any of the claim 22-29 or 31-44, for a medical use, in particular for a use in the treatment of a pervasive development disorder, a neurogenetic syndrome associated with a pervasive development disorder or a symptom of a pervasive development disorder.

46. Supplement, food product or kit of parts according to claim 45, for use in a daily dosage of the sum of histidine, lysine, threonine and tryptophanin the range of 1-20 g, in particular in the range of 2-10 g.
